(19) United States
(12) Patent Application Publication
(10) Pub. No.: US 2023/0302233 A1
(43) Pub. Date: Sep. 28, 2023

(54) EDTA FOR TREATMENT OF CYSTIC FIBROSIS AND OTHER PULMONARY DISEASES AND DISORDERS

(71) Applicant: SterileCare Inc., Markham (CA)

(72) Inventor: Karen Mueller, Newmarket (CA)

(21) Appl. No.: 18/127,175

(22) Filed: Mar. 28, 2023

Related U.S. Application Data
(60) Provisional application No. 63/324,332, filed on Mar. 28, 2022.

(51) Int. Cl. A61M 11/00; A61K 31/198; A61P 11/00; A61K 9/00

(52) U.S. Cl. CPC A61M 11/006; A61K 31/198; A61P 11/00; A61K 9/0078

(57) ABSTRACT

Compositions, solutions, products, devices, and methods related to EDTA, and uses thereof in treating pulmonary diseases and disorders such as cystic fibrosis are disclosed. Also disclosed are an inhalation device and an EDTA composition for dispensing via the inhalation device.

### CROSS-REFERENCE TO RELATED APPLICATION

This application claims benefit of U.S. Provisional Patent Application No. 63/324,332, which was filed in the U.S. Patent and Trademark Office on Mar. 28, 2022, the entire contents of which is incorporated herein by reference for all purposes.

### FIELD OF THE DISCLOSURE

The present disclosure is generally related to compositions, solutions, and methods of making, using, and testing products comprising EDTA adapted for pulmonary treatment of patients suffering from pulmonary diseases and disorders, including cystic fibrosis.

### BACKGROUND

Cystic fibrosis is a genetic disease characterized by impaired mucociliary clearance in the lungs, due in part to elevated viscosity or viscoelasticity of the airway surface liquid (ASL) making it more difficult for the cilia to clear mucous from the lungs. This often promotes biofilm formation by a variety of bacteria that can further harm the health of the patient. (On the other hand, when the viscosity/elastoviscosity of ASL is too low, the effect of gravity pulling ASL downward may not be overcome by the cilia and then fluid can accumulate in the lungs.) There have been many efforts to cope with cystic fibrosis, but no cure is available and treatments offer suffer from relatively low success.

Cystic fibrosis and chronic obstructive pulmonary disease (COPD) are pulmonary disorders involving chronic airway infections in which biofilms can play a serious role. The bacteria commonly associated with these illnesses are often multidrug-resistant pathogens and may include strains of Pseudomonas aeruginosa (PA), Acinetobacter baumannii, Staphylococcus aureus and Burkholderia cepacia. There is also a need for improved treatments for one or more of these ailments that can help cope with such infections and help remove the associated microbial biofilms in the lungs.

Another class of pulmonary disease of note is pulmonary fibrosis, in which lung tissue becomes scarred and stiff. Idiopathic pulmonary fibrosis (IPF) is a progressive form of the disease of unknown cause. Various oral or inhaled medications have been attempted in the treatment of these and other pulmonary diseases. In these diseases and several others, there is a need for improved formulations for application to the lungs by nebulizers, inhalers, or other devices.

Current methods of treating cystic fibrosis and IPF include use of N-acetyl cysteine (NAC), which is often used to mitigate cystic fibrosis due to its mucolytic effect that can assist with removal of the mucous/biofilm material in the lungs, whereas for IPF its role as an antioxidant has been proposed as a mechanism for the limited benefits seen with NAC treatment.

However, previous treatment methods suffer from a number of challenges and improved treatments are needed. Such challenges include limited efficacy (IPF remains a fatal disease and the benefits from prior treatments are generally minor for many patients), difficulties in dispensing the medication efficiently, the tendency for many seemingly successful drugs to have little efficacy with numerous patients, etc. There is a need for improved methods of treatment, improved formulations, improved test methods, improved dispensing methods and devices, improved methods of monitoring or adjusting delivery to be effective, improved methods of diagnosis, etc.

A variety of other diseases face similar challenges and may benefit from improved medicaments delivered to the lungs or improved systems and methods for treatment. For example, for premature infants, bronchopulmonary dysplasia (BPD) often poses serious risks. Ventilator induced lung injury (VILI) is also a serious challenge, including patients with severe COVID or pneumonia. Other diseases of note include pneumonia, emphysema, bronchitis, and various viral infections such as severe acute respiratory syndrome coronavirus (SARS-COV), Middle East respiratory syndrome coronavirus (MERS-COV), SARS-CoV-2 (COVID-19), etc.

Statements about the unmet needs in the fields considered herein are given by way of background only. In the disclosure below, while it is believed that various aspects described may address one or more of the needs listed above, it is not necessary for any or all such needs to be specifically addressed in any of the claims.

### SUMMARY

Provided herein is a system comprising an inhalation device and a composition for dispensing via the inhalation device. The composition may comprise at least about 0.5% (w/w) of EDTA and a pharmaceutically acceptable carrier or excipient. The composition may comprise about 0.5-15% (w/w) EDTA. The pharmaceutically acceptable carrier may be a saline solution. The saline solution may have a pH of about 7.5-11.3. The saline solution may have a pH of about 7.3. The composition may comprise about 5-15% NaCl. The saline may comprise about 1.5-13% NaCl. The saline solution may comprise about 0.9% NaCl. The composition may comprise an alkaline compound, which may be NaOH. The composition may comprise N-acetyl cysteine.

The inhalation device may be a nebulizer or an inhaler. The inhaler may be a standard metered dose inhaler, breath-activated inhaler, or a dry powder inhaler. The inhaler may be a dry powder inhaler, and the composition may comprise nanoparticles, which may comprise a polymer. The polymer may be one or more of polylactic acid (PLA), poly(lactic-glycolic acid) (PLGA), polyurethane (PU), poly(methyl methacrylate), polyester, polyvinylpyrrolidone (PVP), silicone rubber, and polyvinyl alcohol. The polymer may be polyvinyl alcohol.

Provided herein is a system comprising a nebulizer and a composition for dispensing via the nebulizer. The composition may comprise at least about 0.5% (w/w) EDTA in a saline solution. The pH of the solution may be about 7.5-11.3.

Provided herein is a system comprising an inhaler device and a composition for dispensing via the inhaler device. The composition may comprise at least about 0.5% (w/w) EDTA in a saline solution. The pH of the solution may be about 7.5-11.3. The inhaler device may be a standard metered dose inhaler or a breath-activated inhaler.

Provided herein is a system comprising a dry inhaler device and a composition for dispensing via the dry inhaler device. The composition may comprise at least about 0.5% (w/w) EDTA contained in microparticles or nanoparticles. The microparticles or nanoparticles may comprise at least one polymer, which may be polyvinyl alcohol.

Provided herein is a method of treating a pulmonary disease or disorder in a subject in need thereof, which may comprise administering to the subject a composition comprising EDTA. Also provided herein are use of the composition comprising EDTA in the manufacture of a medicament for treating the pulmonary disease or disorder, and the composition comprising EDTA for treating the pulmonary disease or disorder. The composition may be administered via the inhalation device. The pulmonary disease or disorder may be cystic fibrosis, pulmonary fibrosis, idiopathic pulmonary fibrosis, chronic obstructive pulmonary disease (COPD), pneumonia, bronchopulmonary dysplasia (BPD), ventilator-induced lung injury (VILI), Coronavirus Disease 2019 (COVID-2019), pneumonia, emphysema, bronchitis, or a pulmonary infection. The pulmonary infection may be by a bacterium, a virus, a fungus, or a Mycobacterium. The virus may be severe acute respiratory syndrome coronavirus (SARS-COV), Middle East respiratory syndrome coronavirus (MERS-COV), or SARS-CoV-2 (COVID-19).

### BRIEF DESCRIPTIONS OF THE DRAWINGS

The accompanying drawings illustrate various embodiments of systems, methods, and various other aspects of the embodiments. Any person with ordinary art skills will appreciate that the illustrated element boundaries (e.g., boxes, groups of boxes, or other shapes) in the figures represent an example of the boundaries. It may be understood that, in some examples, one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings.

FIG. 1 depicts a nebulizer apparatus that may be used in various aspects disclosed herein.

FIG. 2 is an exploded view of another version of a nebulizer apparatus that may be used in various aspects disclosed herein.

FIG. 3 depicts an inhaler that may be used in various aspects disclosed herein.

FIG. 4 depicts a version of a depicts a drug delivery control system for use with certain aspects of the systems, methods, formulations, and devices disclosed herein.

### DETAILED DESCRIPTION

The present disclosure involves formulations such as solutions comprising one or more salt(s) of ethylene diamine tetraacetic acid (EDTA) that may be at a prescribed concentration and/or pH, and may be provided as methods or systems for treatment of pulmonary diseases and disorders, or be combined with devices for delivery of such formulations.

The EDTA may be combined with a saline solution to give a medicament solution with saline at or near the physiological concentration (0.9%) or at a hypertonic concentration such as from about 1.5% to 13%, about 1.5% to 10%, about 1.5% to 7%, about 1.5% to 3%, about 2% to 7%, etc. In another aspect, the EDTA composition is suitable for inhalation as a dry powder, which may be a microcrystalline or nanocrystalline form known in the art.

In general, when exemplary ranges are given for a parameter, it should be understood that the choice of lower and upper range limits can freely be selected from any of the limits given, or indicated increments therebetween. Thus, an example of range limits of from 1 to 20, 2 to 15, 3 to 10, and 1.5 to 10 should be understood to also provide support for a lower limit from any of 1, 1.5, 2, 2.5, 3, 3.5, . . . 10, etc.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL.” The endpoint may also be based on the variability allowed by an appropriate regulatory body, such as the FDA, USP, etc.

Without wishing to be bound by theory, the inventors propose that certain properties of EDTA, such as its antiseptic properties and/or its ability to bind iron and/or calcium or other metals (e.g., the Ca²⁺ and Mg²⁺ in the lipopolysaccharides of certain bacterial membranes) may play a role in preventing biofilm formation in the lungs or assist in removing it. Such mechanisms may help it provide benefits for patients suffering from pulmonary disorders such as cystic fibrosis, IPF, COPD, pneumonia, lung disorders due to COVID, etc., particularly when applied to the lungs as an aerosol or by other inhalation means. For the case of cystic fibrosis, for example, it is proposed, again without wishing to be bound by theory, that synergy of mechanisms is achieved at certain elevated pH ranges due to the ability of EDTA to more effectively undermine biofilm materials and the ability of the elevated pH of the associated aerosol liquid to reduce the viscosity of the ASL in the lungs of cystic fibrosis patients.

Further synergies may be achieved in combination with other factors such as hypertonic saline solution and the presence of a second medicament. Such synergistic combinations may include EDTA combined with or administered in conjunction with one or more additional therapeutic agents such as an antioxidant, a mucolytic agent or mucous thinning drug such as NAC, an anti-inflammatory compound, nitric oxide or a nitric-oxide releasing agent such as a nitrite (here NO gas may be administered directly in conjunction with the administration of EDTA and may be via the same or a different delivery route), bronchodilators, corticosteroids, phospholipids such as dipalmitoyl phosphatidyl choline, surfactants such as natural or synthetic lung surfactants (dipalmitoyl phosphatidyl choline being a major component of natural lung surfactant), and one or more antibiotics such as taurolidine, tobramycin, ceftazidime, co-trimoxazole, etc. In some aspects, EDTA may be provided in a solution that also comprises one or more of the other therapeutic agents such that both agents are provided via the same route such as by inhalation of the aerosol from a nebulizer or inhaler, but in other aspects another therapeutic agent may be provided via a separate route, such as oral ingestion or intravenous delivery of an antibiotic, mucolytic agent, etc., in which case the EDTA is understood to be administered in conjunction with the other therapeutic agent even though it is not physically in the same solution.

Administering the EDTA may be within a fixed temporal period before or after the other therapeutic agent is delivered, such as both occurring on the same day, or within 4 hours, 2 hours, 1 hour, or 30 minutes of each other, for example. When the two agents are not both present in the same solution, they may be provided in containers that are grouped or connected together as part of a system or kit to facilitate the combined use of the agents together for treatment of the pulmonary disorder.

Provided herein is a composition comprising at least one salt of ethylene diamine tetraacetic acid (EDTA) in solution, with an EDTA concentration of at least about 0.5% (w/w), such as from about 0.5% to 15%, about 1% to 12%, about 2% to 10%, about 1% to 8%, etc. The composition may be an aqueous solution. In another aspect, the aforementioned concentration ranges refer to the combined concentration of the tri-sodium salt or tetra-sodium salt of EDTA. The pH of the aqueous composition may be any suitable range such as from 5 to 11. However, since cystic fibrosis patients tend to have airway surface liquid (ASL) at a significantly lower pH than those without cystic fibrosis (e.g., less than 6 compared to over 7), and since lower pH tends to increase the viscosity or elastoviscosity of ASL, in some aspects, it may be helpful to apply an aqueous composition at a pH near or above the normal pH of about 7.3 for ASL.

Thus, the pH of the aqueous composition may be about 7 or higher or about 7.3 or higher, such as from about 7 to 15, about 7 to 11.5, about 7.3 to 15, about 7.3 to 11, about 7.5 to 10.5, about 7 to 9.8, about 7 to 8.5, about 7.7 to 9.5, about 7.8 to 9.7, about 8 to 11, and about 8.3 to 10.8. The pH may simply be adjusted by titration with an alkaline compound such as sodium bicarbonate (NaHCO₃), sodium carbonate (NA₂CO₃), sodium hydroxide (NaOH), or related salts such as potassium carbonate, etc., or may be buffered with pharmaceutically acceptable buffering agents to more effectively help elevate the pH of the ASL in the lungs. In one example, the composition comprises EDTA and NaOH.

Mucous thinning agents may include dornase alfa (also known as rhDNase Pulmozyme®), hypertonic saline, mannitol dry powder, mannitol solution, NAC, albuterol, etc. Anti-inflammatory agents may include azithromycin (an antibiotic that has anti-inflammatory properties) and ibuprofen. Pulmozyme®, for example, is provided in single-use ampules with 2.5 mL of the solution to placed in a nebulizer bowl. Each mL of aqueous solution contains 1 mg dornase alfa, calcium chloride dihydrate (0.15 mg) and sodium chloride (8.77 mg). The nominal pH of the solution is 6.3. Typically one or two ampules may be used daily.

Bronchodilators may include albuterol (also known as salbutamol, and marketed as VENTOLIN®, PROAIR®, PROVENTIL®), levalbuterol hydrochloride (XOPENEX®) or levosalbutamol, terbutaline (BRONCLYN®, BRETHINE®, BRICANYL®, BRETHAIRE®, MAREX®, and TERBULIN®), pirbuterol (MAXAIR®), epinephrine (PRIMATENE® Mist), racemic epinephrine (ASTHMANEFRIN™), ephedrine (Bronkaid), and long-acting bronchodilators such as salmeterol (SEREVENT®, SERETIDE®), clenbuterol (SPIROPENT®), formoterol, bambuterol, and indacaterol.

Pharmaceutically acceptable antioxidants may include water soluble antioxidants, such as NAC, ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxy toluene (BHT), lecithin, propyl gallate, a-tocopherol, and the like; and metal chelating agents, such as citric acid, sorbitol, tartaric acid, phosphoric acid, and the like.

Corticosteroids may be in any form, such as aerosol liquid (ready for use in a nebulizer, for example, or in conjunction with an inhaler), aerosol powder (also for use in nebulizer system or powder inhalers), powder, and suspension, and may be delivered as an aerosol for inhalation or delivered through oral ingestion, intravenous delivery, etc. Corticosteroids include glucocorticoids such as cortisol (hydrocortisone), prednisolone and cortisone, and mineralocorticoids such as aldosterone and the synthetic Fludrocortisone (Florinef). Steroids may be used, including oral steroids at a prednisolone-equivalent dose of 0.5 to 4 mg/kg applied daily, on alternate days, or less frequently, as desired. Steroids may also include any of those used for asthma therapy such as beclomethasone (ASMABEC®, CLENIL MODULITE®, and QVAR®), budesonide (EASYHALER BUDESONIDE®, NOVOLIZER BUDESONIDE® and PULMICORT®), Ciclesonide (ALVESCO®), fluticasone (FLIXOTIDE®), mometasone (ASMANEX TWISTHALER®), etc. Steroids may also be combined with bronchodilators, such as the combinations marketed as FOSTAIR® (formoterol and beclometasone), SERETIDE® (salmeterol and fluticasone), and SYMBICORT® (formoterol and budesonide).

Other useful adjuvants may be present in the aqueous composition such as pharmacologically acceptable surfactants, humectants, etc., including glycine, citric acid, sorbitol, tartaric acid, etc.

The EDTA may be administered in combination with mechanical methods to assist mucous clearance from the lungs, such as chest physical therapy (CPT) that may include a combination of techniques several times a day such as clapping with cupped hands on the front and back of the chest, certain breathing and coughing techniques (e.g., autogenic drainage or active cycle of breathing techniques [ACBT]), vigorous exercise, use of mechanical devices such as a tube the patient blows into (a positive expiratory pressure [PEP] device) and a machine that pulses air into the lungs (e.g., a vibrating vest).

Soluble salts of EDTA are used in compositions of the present invention. Sodium salts of EDTA are commonly available and generally used, including di-sodium, tri-sodium and tetra-sodium salts, although other EDTA salts, including ammonium, di-ammonium, potassium, di-potassium, cupric di-sodium, magnesium di-sodium, ferric sodium, and combinations thereof, may be used, provided they are pharmacologically acceptable and efficacious. Various combinations of EDTA salts may be used and may be preferred for particular applications.

In one embodiment, aqueous compositions comprising one or more sodium salt(s) of EDTA at a pH higher than physiological pH are provided as nebulizing or inhalant compositions.

Thus, in one aspect, a system is disclosed, which may be for treating a pulmonary disease or disorder. The system may comprise a nebulizer and an EDTA composition disclosed herein for dispensing via the nebulizer. The medicament may comprise a saline solution further comprising at least 0.5% (w/w) of EDTA at a pH from about 7.5 to 11.3. In related aspects, the saline solution comprises about 0.9% or more of sodium chloride, or from about 1.5% to 23% sodium chloride, or from about 3% to 15% sodium chloride, or from about 5% to 15% sodium chloride. In any of the above aspects, the EDTA concentration in the saline solution may be from about 1% to 10%. Likewise, in any of the above aspects, the concentration of tri-sodium and tetra-sodium EDTA in the saline solution may range from about 1% to 10%.

In any of the above aspects, the medicament of the system may further comprise N-acetyl cysteine, such as at least about 0.5% NAC in the saline solution, or may further comprise any one or more of the following, in any combination: a bronchodilator, an effective amount of nitric oxide or of a source of nitric oxide, an effective amount of a corticosteroid, an effective amount of an anti-inflammatory compound, an effective amount of an antibiotic such as at least one of taurolidine, tobramycin, ceftazidime, and co-trimoxazole, etc. In any of the above aspects or combinations, the pH of the saline solution may be greater than about 7.5 such as from about 8 to 11.3, about 8.5 to 11, about 9 to 11, etc.

In another aspect, a method is provided for treating a pulmonary disease or disorder. The pulmonary disease or disorder may be one or more of cystic fibrosis, pulmonary fibrosis, idiopathic pulmonary fibrosis (IPF), chronic obstructive pulmonary disease (COPD), pneumonia, bronchopulmonary dysplasia (BPD), ventilator-induced lung injury (VILI), Coronavirus Disease 2019 (COVID-2019), pneumonia, emphysema, bronchitis, and a pulmonary infection. The pulmonary infection may be by a bacterium, a virus, a fungus, or a Mycobacterium. The virus may be severe acute respiratory syndrome coronavirus (SARS-COV), Middle East respiratory syndrome coronavirus (MERS-COV), or SARS-CoV-2 (COVID-19). The bacterium may be Pseudomonas aeruginosa (PA), Acinetobacter baumannii, Staphylococcus aureus, or Burkholderia cepacian. The method may comprise administering an EDTA to a subject in need thereof via a delivery system disclosed herein. In one example, the method comprises delivery of an effective quantity of liquid droplets to the lungs, the droplets comprising a saline solution and at least about 0.5% by weight of EDTA at a pH of from about 7.5 to 11.3 or any other previously cited pH range. In any of such aspects, the volume of liquid droplets applied to the lungs (e.g., about 5 ml or more per hour, such as about 10, 20, 30, 40, or 50 ml per hour) may be efficacious in elevating the pH of the mucous in the lungs, as measured with a test of sputum, by at least 0.5 pH units, and is effective in reducing the viscosity of mucous in the lungs, such as by reducing viscosity by at least about 10%, 20%, 40%, or 80%.

As used herein, the terms “subject,” “individual,” and “patient” are used interchangeably. None of the terms are to be interpreted as requiring the supervision of a medical professional (e.g., a doctor, nurse, physician's assistant, orderly, hospice worker). As used herein, the subject may be any animal, including a mammal (e.g., a human or non-human animal) or a non-mammal. In one embodiment, the subject is a human.

As used herein, the terms “treat,” “treating”, or “treatment,” and other grammatical equivalents, include ameliorating the underlying causes of one or more symptoms of a disease or condition; alleviating, abating, or ameliorating one or more symptoms of a disease or condition; ameliorating or reducing the appearance, severity, or frequency of one or more symptoms of a disease or condition; inhibiting the disease or condition, such as, for example, arresting the development of the disease or condition, relieving the disease or condition, causing regression of the disease or condition, relieving a condition caused by the disease or condition, or inhibiting the symptoms of the disease or condition either prophylactically and/or therapeutically. Methods of treatment as disclosed herein include disclosures of the use of the compositions provided herein for the treatment of any indication described herein, and include disclosures of the compositions provided herein for the use in treating any indication described herein.

In another aspect, an aerosol liquid is disclosed for delivery to the lungs of a pulmonary patient, the liquid comprising saline solution, at least about 0.5% EDTA, and at least about 0.5% N-acetyl cysteine, having a pH of about 7.5 to 11.3, the aerosol liquid being disposed in an inhalation device.

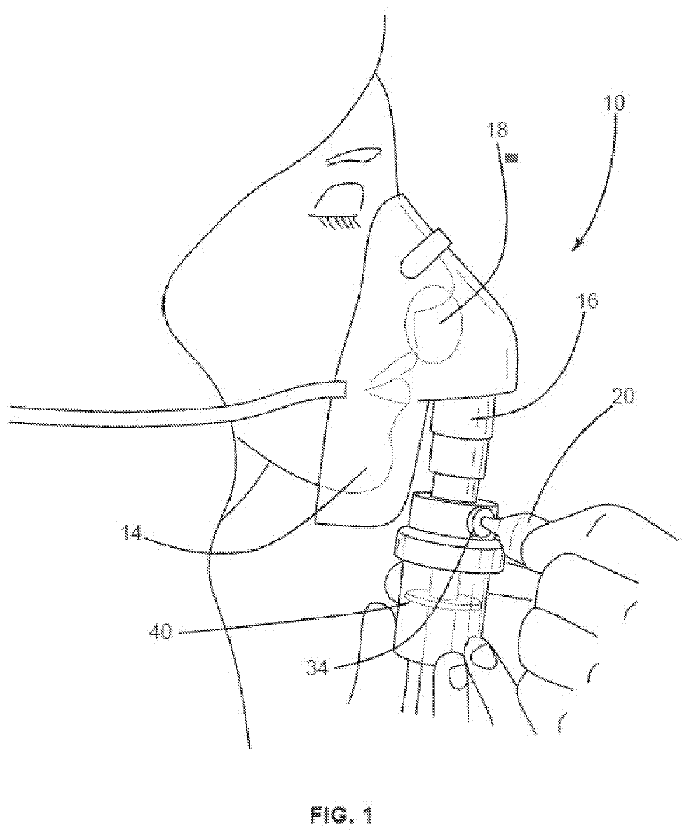

FIG. 1 depicts a version of a nebulizer apparatus 10 that may be used with the aerosol liquid compositions comprising EDTA disclosed herein for treatment of cystic fibrosis or other pulmonary disorders. In this embodiment, the medication reservoir 40 comprises a rapid injection port 34 disposed directly in the reservoir 40 which may be accessed by a drug dispenser 20, which may allow additional medicament to be placed into the medication reservoir 40 by squeezing, pumping, gravitational flow, etc. Thus, the rapid injection port 34 may be in fluid communication directly with the internal volume of the medication reservoir 40 to allow direct deposit of the medication from the dispenser 20. The reservoir 40 is also coupled to a face mask 14 via a mask connector 16 disposed substantially beneath the nasal portion of the face mask 14. The face mask 14 also comprises a plurality of exhaust ports 18 also defined in the nasal portion of the face mask 14. To use the nebulizer apparatus 10, the face mask 14 is placed over the nose and mouth of a patient with the reservoir 40 coupled to the mask connector 16 of the face mask 14. The medicated aerosol flows from the medication reservoir 40 through the hollow mask connector 16 and to the face mask 14 of the nebulizer apparatus 10. Upon inhalation or exhalation by the patient, the medicated aerosol flowing to the face mask 14 is then delivered to the patient through their nose or mouth. The exhalation of the patient is removed from the mask 14 by flowing through the exhaust ports 18. The medication reservoir 40 may be coupled to the mask connector 16 via a ball and socket configuration. Such a ball and socket connection may allow for effective delivery of medicated aerosol to patients lying on their backs, ensuring that the medication reservoir 40 is maintained in a substantially vertical orientation independent of the position of the patient. As a result, the liquid medication is properly aerosolized and delivered to the patient, avoiding spillage of the liquid medication.

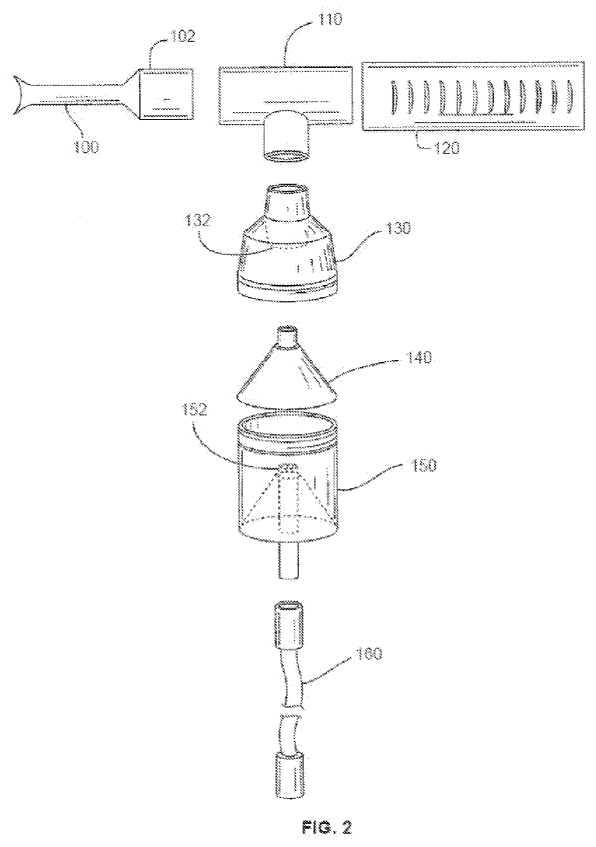

FIG. 2 depicts an exploded view of the components of a commonly used nebulizer system, shown here as a kit of seven plastic pieces which are assembled prior to use to provide for delivery of the medication to a patient via inhalation. There is a mouthpiece 100 with a connecting portion 102 that fits onto one end of a T connector 110. Similarly, the other end of the T connector 110 is attached to a flex tube 120, typically by force fit. The parts generally can be assembled and disassembled with a simple twisting action but when pressed together, form a substantially airtight seal. The bottom part of the T connector 110 is connected to a cup cover 130. The cup cover 130 has a screen 132 that screens the material going into the T connector. There is a cup 150 for receiving the medicine to be nebulized. The cup also has a venturi 152 projecting through the bottom.

In a typical use, a vial containing the medication for administration through the nebulizer is opened and poured into the cup 150 where it accumulates at the edges of the rounded bottom of the cup. The venturi 152 is surrounded by a conical plastic piece through which it passes. The shape of the conical piece of the medicine cup 150 matches substantially the shape of the venturi cover 140. Once the medicine is poured into the cup, the venturi cover 140 is placed over the venturi 152 and the filled medicine cup is screwed, using threaded portions on each piece, onto the cup cover 130. In this way, the medicine is held in place ready for administration. In this case, the medicine (not shown) comprises a saline solution such as a hypertonic saline solution (e.g., from 3% to 12% sodium chloride) with at least 0.5% by weight of EDTA at a pH greater than 7.5, such as from 7.8 to 11.3, and optionally comprising an effective concentration of NAC, a bronchodilator, a mucous thinning drug, or other active agents as described herein.

In use, the bottom of the airline feeding the venturi in the medicine cup is attached to an air hose 160, to which is applied to a source of air pressure thus activating airflow through the venturi. By venturi action, the exhaust of the air flow through the small opening of the venturi results in a reduction in pressure on the downstream side of the airflow so that the medicine from the medicine cup is fed under positive pressure up in the interstices between the conical shape of the medicine cup and the venturi cover and is exhausted then through the screen 132 into the bottom of the T connector 110.

A patient is asked to inhale the aerosol mist provided through the cup cover screen into the airflow channel between the mouthpiece 100 and the flex tube 120. As a patient takes the mouthpiece 100 in their mouth, and inhales, air flows through the open end of the flex tube 120, through the T connector 110, picking up the aerosol medication and into the patients' air passages through the mouthpiece 100.

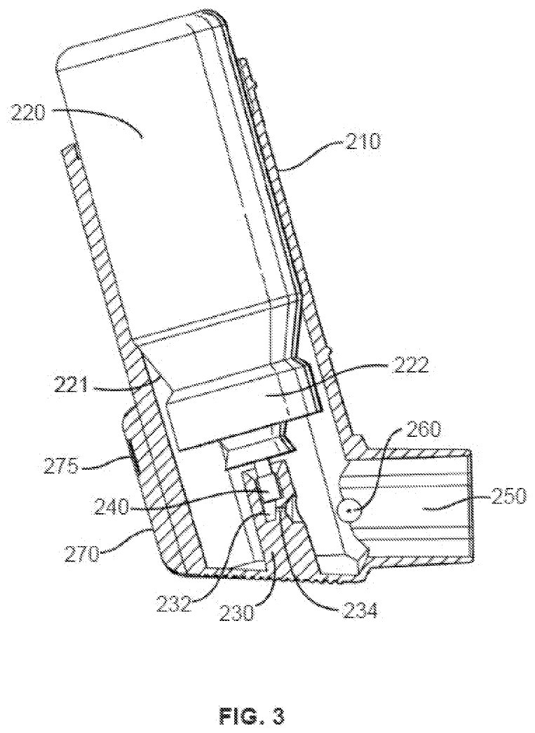

FIG. 3 depicts an inhaler 200. As shown in the cross-sectional view, an aerosol canister 220 is located in the housing 210 so that one end protrudes from its open top, the canister being positioned such that the neck 221 and valve ferrule 222 are enclosed within housing 210. The canister may comprise an aqueous solution of EDTA optionally with one or more other active ingredients, at a suitable pH and concentration as described herein, and may be pressurized with a non-reactive gas that may comprise an effective concentration of nitric oxide gas, when desired.

A support 230 is provided at the lower end of the housing 210 and has a passage 232 in which the valve stem 240 of the aerosol canister 220 can be located and supported. A second passage 234 is provided in the support 230 and is directed towards the interior of the outlet 250. An emitter 260 is located on outlet 250. Emitter 260 emits an infra-red beam (not shown) across outlet 50 onto a detector (not shown) attached to the other side of outlet 250. The emitter 260 may emit a continuous beam of infra-red radiation or may emit a pulsed beam at varying intervals and for varying duration. The emitter 260 is under the control of a microprocessor within dose counter unit 270. The detector (not shown) is responsive to change in infra-red radiation and generates a signal readable by the microprocessor. When the level of energy falls below a predetermined threshold for a predetermined period (calibrated to respond to an emission of medicament released from canister 220) the microprocessor updates the display on the visual display unit 275 indicating the doses used or remaining within the canister.

The detector is calibrated to respond to an emission of medicament released from canister 220. The signal results in a change in the display on the visual display unit 275 indicating the doses used or remaining within the canister.

Thus, when the parts are in the positions shown in FIG. 3, the protruding portion of the aerosol canister 220 can be depressed to move the canister relative to the valve stem 240 to open the valve and a dose of medicament contained in the canister 220 will be discharged as a medicament release through passage 234 and into the outlet 250 from which it can be inhaled by a patient. One dose will be released from the aerosol canister each time it is fully depressed. The medicament release will interfere with the beam of infra-red radiation from emitter 260 resulting in a reduction in radiation reaching the detector. The visual display unit 275 will be updated to display the number of doses used or remaining within the canister 220. This display may consist of a numerical read out, giving the precise number of doses used or remaining. Alternatively, the display may be indicative of the number of doses used or remaining, based upon a series of colored lights (e.g. green, orange, red) representing an estimated range of the number of doses used or remaining.

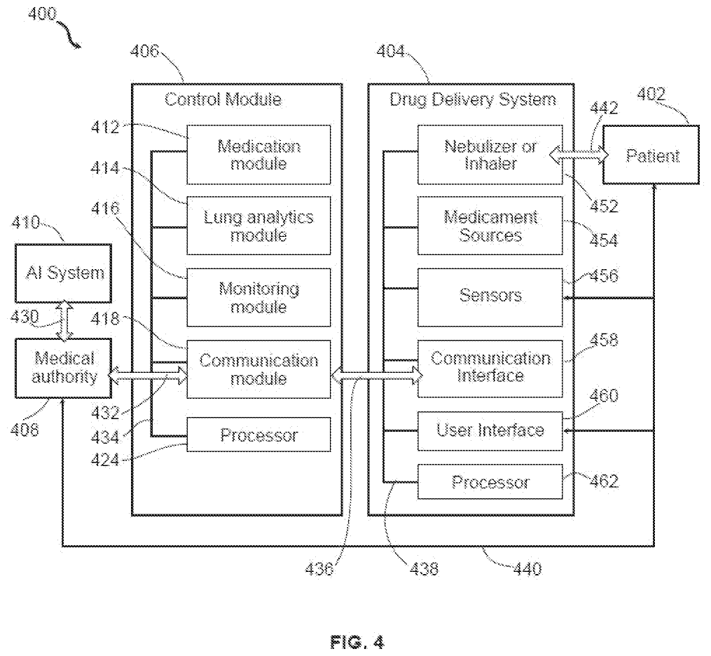

FIG. 4 depicts a drug delivery control system 400 with a drug delivery system 404 in which a nebulizer or inhaler 452 (an inhalation device) provides delivery a medicament 442 from medicament sources 454 to a patient 402. The drug delivery system 404 comprises medicament sources 454 that may comprise ingredients for a customized formulation of an aerosol liquid for delivery via the nebulizer or inhaler 452. Such ingredients may comprise one or more saline solutions or a base liquid to which sodium chloride and other salts may be added as desired to provide hypertonic solutions as desired and may comprise EDTA in powder or solution form that can be used to customize the EDTA concentration of the aerosol liquid. It may further comprise buffering agents to tailor the pH to a desired range. The medicament sources 454 are combined as desired (e.g., as prescribed by the medical authority 408 or in accordance with rules approved by the medical authority 408) with a blending unit (not shown) to provide a tailored aerosol liquid (not shown) at an appropriate pH, temperature, salinity, and with appropriate concentrations of one or more medicaments to the nebulizer or inhaler 452. Automated tools can be used to order and supply materials to replenish supplies of the medicament sources 454.

The drug delivery system 404 further comprises or is associated with sensors that may be used to monitor patient breathing, blood oxygen level, body temperature, lung performance (spirometry and other measures), exhalation gas characteristics including VOC analysis, carbon dioxide levels, etc., body temperature, pulse, blood pressure, pH of ASL, ASL rheological properties, air flow, drug consumption rate or volume remaining, coughing instances, patient comments, etc. Such data can be shared via a communication interface 458 allowing transfer of data 436 to the communication module 418 of the control module 406. Information within the drug delivery system 404 may be shared between components via a bus 438 or other communication means.

The drug delivery system 404 further comprises a user interface 460 that may assist the patient 402 by providing directions, showing progress in the treatment, notifying the patient of completion of a cycle of the regimen, providing feedback on progress being made and on status, etc. It may also provide means for the user to pause therapy, adjust conditions, tailor operation within allowable limits, etc., or to also communicate with a medical authority 408 to make further adjustments or receive further guidance. The drug delivery system 404 also may comprise a processor 462 to enable various actions of the components therein.

The drug delivery system 404 is regulated by a control module 406 comprising a processor 424, a communication module 418 for communicating with a medical authority 408 or with the drug delivery system 404, and a monitoring module 416 for receiving, storing, and interpreting data, in real time when feasible, regarding the therapy being provided such as the medicament being provided, the dose delivered, the target dose, etc. The control module 406 also comprises a lung analytics module 414 for interpreting data related to lung performance and the health of the patient 402, in cooperation with the monitoring module 416 and the transfer of data 436 from the drug delivery system 404, particularly data from the sensors 458 but this may also include input directly from the patient 402 via the user interface 460, which may query the patient 402 periodically on issues related to patient condition. A medication module 412 can assist in the selection of dosage, the selection of the medicaments to use and details of the formulation provided to the nebulizer or inhaler 452 to ensure that the proper concentrations, pH, salinity, etc. are being provided. The medication module 412 is adapted to interact with the medical authority 408 and optionally with the associated AI system 410 to enhance the usefulness of the medication regimen.

A communication channel 440 also may exist directly between the medical authority 408 and the patient, either via digital or electronic means such as telecommunication or personal interaction. The medical authority 408 may have access to some aspects of the user interface 460 or to some aspects of the sensors 456 to assist in supervision of the patient 402, optionally with further supervision and input from the AI system 410.

Thus, in one aspect, a system is provided for monitoring treatment of a pulomonary disorder such as cystic fibrosis, comprising a drug delivery module for delivering a medicament to a patient via inhalation and optionally one or more additional delivery routes, wherein the medicament delivered via inhalation comprises a saline solution with an effective amount of EDTA, the system further comprising a control module adapted to receive information from the patient pertaining to lung status and in response to the received information, to modify the drug delivery module to adjust at least one of the dose of EDTA, the pH of the saline solution, or the concentration of a second active ingredient selected from one of a mucous thinning agent, a bronchodilator, an anti-inflammatory agent, a surfactant, and a steroid, the control module further being in communication with a medical authority to oversee the performance of the system.

### Nebulizers

A wide variety of devices adapted to provide a stream of aerosolized liquid or mist are known as nebulizers, and they are generally adapted to provide means for a medicament to be delivered to the lungs of a patient via the aerosol or entrained particles, and thus can be adapted for use according to many of the aspects disclosed herein. For example, nebulizer types, that may include ultrasonic nebulizers, which generally have a rapidly vibrating piezoelectric crystal that may vibrate at 1.6-3 MHz, which in turn can transfer energy to a liquid medium to produce small droplets such as those having a size range of from about 1 micron to 6 microns. The liquid is often provided from an overlying reservoir.

Jet nebulizers employ a compressed gas source to create the aerosol. The venturi effect pulls liquid into the jet of gas, shearing the fluid and creating fine droplets as the fluid is atomized. One embodiment may use a nebulizer/inhaler which has an inhaler-like delivery system associated with a face mask. The delivery of medicament automatically ceases as the patient exhales. The medicament is provided within a replaceable cartridge.

One system that may be adapted for the purposes herein is liquid, flowable formulations of medicaments packaged in individual dosage unit containers which are interconnected to form a cellular array integrated into a dispensing device capable of individually opening the dosage unit containers and aerosolizing the contents through a nozzle for delivery to a patient. The cellular array is comprised of a plurality of containers with each container having an opening(s) thereon from which a drug-containing formulation may be aerosolized. The dispensing device is a hand-held, self-contained, portable device comprised of a means for removing covers from the containers and automatically dispensing the formulation from individual containers, preferably in response to a signal obtained as a result of measuring the inspiratory flow of a patient. The cellular array is loaded into the dispensing device to form a system which can be used in a method of delivering drugs to a patient via the intrapulmonary route. In one version each container includes an opening covered by a membrane having a plurality of pores therein wherein the pores have a diameter of about 0.5 microns to 50 microns and the dispensing device includes a vibrating device which creates a vibration frequency such that formulation forced through the pores is aerosolized to particles having a diameter of about 1 micron to 100 microns.

This can be adapted by coupling pair of sealed cells to the nebulizing device, with each pair of sealed cells comprising a first sealed cell with sterile nebulizing liquid such as saline water with other optional additives and a second sealed cell containing a powder or a concentrated liquid comprising EDTA and/or other materials for treatment of pulmonary conditions, wherein the powder, when used, may be suitably flowable to flow under the force of gravity into the nebulizing chamber to be combined with the nebulizing liquid. Both chambers as a pair may be opened at the same time or may be individually opened in any order, such as liquid first and then the powder or vice versa.

Another system that can be adapted for use as described herein includes a main body that has a nebulizer outlet and an air channel in communication with the nebulizer outlet. The main body supports a medicine reservoir and a mesh that engages the medicine reservoir and air channel and vibrates to atomize medicine from the medicine reservoir into the air channel for discharge through the nebulizer outlet. The nebulizer outlet and mesh are configured to be received within the oral cavity of the patient when the nebulizer is in use. This system can be adapted by attaching a removable cartridge to the nebulizer that comprises a medicament chamber containing a medicament in liquid, slurry, or powder form, such as EDTA powder combined with other materials such as NAC powder, etc., which can be opened or unsealed to be combined with a liquid in a liquid chamber in the cartridge, or with liquid already provided in the nebulizer. In either case, unsealing the medicament allows it to mix with a suitable liquid to form a solution or suspension which can then be nebulized to deliver to a user.

Other systems with vibrating or active meshes may deliver a medication that involves an active mesh in an active mesh nebulizer, the active mesh being in contact with a liquid formulation of the medication, and configured to generate a plume of liquid droplets having a particle diameter between 1 and 6 micrometers, directing the plume of particles to a mouth of a patient during an inhalation by a patient; and stopping the plume of particles during the inhalation by the patient such that a substantial majority, or nearly all, of the plume of particles is inhaled by the patient.

In addition to those described above, a variety of other nebulizer components and materials may be adapted for the purposes described herein from one or more sources, including ultrasonic sprayers which generally operate by passing liquid through an orifice of an ultrasound instrument tip. Other ultrasonic sprayers operate by atomizing liquid using piezoceramic film and air/propellant.

In one aspect, which may be suited for long-term care and other scenarios, an inhalant is pumped into a chamber of a nebulizer using a pump such as infusion or syringe pumps.

Nebulizers and inhalers described herein may be cooperatively associated with a monitoring device that can monitor medicament use and history, patient breathing performance, recommended versus actually received dosage, etc., and may also comprise analytical tools to analyze exhaled gases, breathing patterns, sounds from the patient's lungs, blood oxygen levels, spirometry measures, and other parameters associated with the health of the patience and the performance of the treatment regime. The monitoring system may also be adapted to control or adjust the dose of the medicament, and may also adjust the pH, temperature of the aerosol and/or of the air, humidity of the air stream, concentration of the EDTA or other medicaments, etc.

### Inhalers

Inhalers are portable devices for delivering a medication to the lungs, typically used by asthma patients to deliver bronchodilators. In the treatment of asthma, the medication may be classified as relievers (short-acting bronchodilators), preventers (steroid inhalers) and long-acting bronchodilators. Physical devices include the standard metered dose inhaler (MDI), sometimes called an evohaler. The MDI contains a pressurized inert gas that propels a dose of medicine into the airway of the user in each “puff.” Each dose is released by pressing the top of the inhaler. Sometimes these are known as evohalers.

Another type of inhaler is the breath-activated inhalers. Though pressurized, there is no need to press a button to activate delivery of the medication. One example is known as an autohaler. Another example the easi-breathe inhaler. A related class of inhalers the dry powder inhalers. There do not contain pressurized gas. Rather, by breathing in at a mouthpiece, the user can trigger release of a dose of dry powder. Such devices may be known as Accuhalers, clickhalers, easyhalers, novolizers, turbohalers and twisthalers. Various MDIs may be used with “spacer devices” that are installed between the inhaler and the mouth and can holds medicine like a reservoir when the inhaler is pressed.

Dry powder inhalers, such as those adapted to release doses of powder from a strip of blister packaging encasing individual doses, can be adapted to deliver EDTA, or a mixture of powders such as EDTA and NAC, or other agents. In one example, the dry powder comprises nanoparticles, which may comprise a polymer. The polymer may be one or more of polylactic acid (PLA), poly(lactic-glycolic acid) (PLGA), polyurethane (PU), poly(methyl methacrylate), polyester, polyvinylpyrrolidone (PVP), silicone rubber, and polyvinyl alcohol. In one example, the polymer is polyvinyl alcohol. The EDTA may be dissolved in a polymer solution, and the solution may be poured into a water-immiscible non-solvent (e.g., isopropyl alcohol) under continuous stirring until a cloudy suspension is formed. The nanoparticles may be dried, which may be under vacuum, and which may be performed at 50° C. The drying may be performed using a rotary evaporator. The EDTA may be Ca—Na₂ EDTA. The concentration of the EDTA dissolved in the polymer solution may be 0.1-1.0 g/mL. The concentration of the polymer, which may be polyvinyl alcohol, may be 1-5%, and may in particular be 1%. The stirring rate of the EDTA in the polymer solution may be about 600, 800, 1000, 1200, 1400, or 600-1400 rpm. The mean particle size of the EDTA nanoparticles may be about 0.3-5.5 μm, and in one example is about 0.3-0.5 μm. The dry powder may also comprise one or more alkaline agents such as buffering agents. In one example, when 1 g of the powder is combined with 10 ml of distilled water at a pH of 7.0, the resulting solution has a pH of 7.5 or higher.

### Test Methods

A variety of tests can be used to assess the lung function of a patient, such as the various aspects measured during spirometry, including forced expiratory volume (FEV), which measures how much air a person can exhale during a forced breath. The amount of air exhaled may be measured during the first (FEV1), second (FEV2), and/or third seconds (FEV3) of the forced breath. Another useful measure from spirometry is forced vital capacity (FVC), the total amount of air exhaled during the FEV test. Other measures that can be applied include total lung capacity (TLC), the volume in the lungs at maximal inflation, the sum of VC and RV; tidal volume (TV), the volume of air moved into or out of the lungs during quiet breathing; residual volume (RV), the volume of air remaining in the lungs after a maximal exhalation; expiratory reserve volume (ERV), the maximal volume of air that can be exhaled from the end-expiratory position; inspiratory reserve volume (IRV), the maximal volume that can be inhaled from the end-inspiratory level; inspiratory capacity (IC), the sum of IRV and TV; inspiratory vital capacity (IVC), the maximum volume of air inhaled from the point of maximum expiration; vital capacity (VC), the volume of air breathed out after the deepest inhalation; ratios such as RV/TLC or FEV1/FVC; alveolar gas (VA) volume; actual volume of the lung (VL) including the volume of the conducting airway; the maximum instantaneous flow achieved during a FVC maneuver (FEFmax); forced inspiratory flow (FIF), wherein measurement of the forced inspiratory curve is denoted analogously to that for the forced expiratory curve, with, for example, the maximum inspiratory flow is denoted FIFmax; peak expiratory flow (PEF), the highest forced expiratory flow measured with a peak flow meter; and maximal voluntary ventilation (MVV), the volume of air expired in a specified period during repetitive maximal effort.

Functional residual capacity (FRC), the volume of air present in the lungs at the end of passive expiration, is not measured in spirometry, but it can be measured with a plethysmograph or dilution tests such as a helium dilution test.

Gas analysis can also be applied to detect the presence of volatile organic compounds (VOCs) associated with specific pathogens or other disorders. An analytical procedure for the detection of VOCs from the headspace of epithelial lung cells infected with four human pathogens was developed. The feasibility of this method was tested in a cystic fibrosis (CF) outpatient clinic in vivo, with samples analyzed by gas chromatography-mass spectrometry (GC-MS). Other test methods to consider include sputum culture texts, biopsies, x-rays, MRIs, and lung ultrasound.

For measurement of the rheological properties of ASL, the viscosity can be measured using the cone and plate viscometer system. In one approach, a constant shear stress of 10 Pa is applied. In some aspects, the measured viscosity of the ASL from a given site in the lungs can be reduced by at least 10% by application of the aerosol liquids, systems, or methods described herein.

Thus, in some aspects, a patient presenting with cystic fibrosis or other pulmonary diseases may have a sample of ASL extracted and measured for pH and viscosity, and evidence of microbial infection may also be obtained via a sputum culture, measurement of VOCs in airway gases, detection of biomarkers, etc., and spirometric or other measures of lung performance may be made as well. In response, a physician may recommend the use of an EDTA medicament via an inhalation device in order to help relieve the pulmonary disorder, such as to reduce viscosity of the ASL, to reduce the impact of biofilm in the lungs, to improve lung performance, etc. Then an aerosol liquid may be prescribed or provide to the patient with a prescribed regimen, such as the use of from 10 ml to 50 ml of the aerosol fluid three times a day via a nebulizer or inhaler, wherein the aerosol liquid may be provided with from 0.5% to 3% EDTA in a hypertonic saline solution (e.g., from 3% to 12% saline) at a pH of from 8.5 to 11 in a pharmaceutically acceptable buffering system, optionally further comprising 0.5% to 1.5% NAC and other medicaments, as desired. After a fixed number of treatments, ASL may again be sampled and tested to confirm that the viscosity has dropped by a desired amount such as by at least 10%, 15%, 20%, etc., up to, say 60% or 80%, and to confirm that other measures of lung performance or infection control show that a benefit has been achieved. The dosage and regimen may then be adjusted for ongoing maintenance. This iterative process may also be assisted with artificial intelligence/machine learning tools to apply comprehensive interpretations of diverse data regarding the patient and maintenance of the patient's health in light of extensive medical knowledge that may be missed by a single practitioner.

### Remarks

When listing various aspects of the products, methods, or system described herein, it should be understood that any feature, element or limitation of one aspect, example, or claim may be combined with any other feature, element or limitation of any other aspect when feasible (i.e., not contradictory). Thus, disclosing one particular pH and saline concentration in one aspect and a particular concentration of taurolidine in another aspect should be understood as intending to also teach the combination of the particular pH, saline concentration, and taurolidine concentration. Likewise, disclosure of particular limitations in a method should be understood to support such limitation, when feasible, in a system or a formulation, etc., and vice versa.

Having described aspects of the invention in detail, it will be apparent that modifications and variations are possible without departing from the scope of aspects of the invention as defined in the appended claims. As various changes could be made in the above compositions, products, and methods without departing from the scope of aspects of the invention, it is intended that all matter contained in the description be interpreted as illustrative, not in a limiting sense.

What is claimed is:

1. A system comprising an inhalation device and a composition for dispensing via the inhalation device, the composition comprising at least about 0.5% (w/w) of EDTA and a pharmaceutically acceptable carrier or excipient.

2. The system of claim 1, wherein the composition comprises about 0.5-15% (w/w) EDTA.

3. The system of claim 1, wherein the pharmaceutically acceptable carrier is a saline solution, wherein the saline solution has a pH of about 7.5 to 11.3.

4. The system of claim 1, wherein the saline solution has a pH of about 7.3.

5. The system of claim 3, wherein the composition comprises about 5-15% NaCl.

6. The system of claim 5, wherein the saline solution comprises about 1.5-13% NaCl.

7. The system of claim 3, wherein the saline solution comprises about 0.9% NaCl.

8. The system of claim 3, wherein the composition further comprises an alkaline compound.

9. The system of claim 8, wherein the alkaline compound is NaOH.

10. The system of claim 1, wherein the inhalation device is a nebulizer or inhaler.

11. The system of claim 10, wherein the inhaler is a standard metered dose inhaler, a breath-activated inhaler, or a dry powder inhaler.

12. A system comprising a nebulizer and a composition for dispensing via the nebulizer, the composition comprising at least about 0.5% EDTA in a saline solution, wherein the pH of the saline solution is about 7.5-11.3.

13. A system comprising an inhaler device and a composition for dispensing via the inhaler device, the composition comprising at least about 0.5% EDTA in a saline solution, wherein the pH of the saline solution is about 7.5-11.3.

14. The system of claim 13, wherein the inhaler device is a standard metered dose inhaler or a breath-activated inhaler.

15. A system comprising a dry inhaler device and a composition for dispensing via the dry inhaler device, the composition comprising at least about 0.5% (w/w) EDTA contained in microparticles or nanoparticles.

16. The system of claim 15, wherein the microparticles or nanoparticles comprise at least one polymer.

17. The system of claim 16, wherein the polymer is polyvinyl alcohol.

18. A method of treating a pulmonary disease or disorder in a subject in need thereof, comprising administering to the subject a composition comprising EDTA via the system of claim 1.

19. The method of claim 18, wherein the pulmonary disease or disorder is selected from the group consisting of cystic fibrosis, pulmonary fibrosis, idiopathic pulmonary fibrosis, chronic obstructive pulmonary disease (COPD), pneumonia, bronchopulmonary dysplasia (BPD), ventilator-induced lung injury (VILI), Coronavirus Disease 2019 (COVID-2019), pneumonia, emphysema, bronchitis, and a pulmonary infection.

20. The method of claim 19, wherein the pulmonary infection is by a bacterium, a virus, a fungus, or a Mycobacterium.

21. The method of claim 20, wherein the virus is severe acute respiratory syndrome coronavirus (SARS-COV), Middle East respiratory syndrome coronavirus (MERS-COV), or SARS-CoV-2 (COVID-19).
